(19) United States
(12) Patent Application Publication
(10) Pub. No.: US 2022/0002811 A1
(43) Pub. Date: Jan. 6, 2022

(54) Prostate Cancer Biomarker Assays

(71) Applicants: Queen's University at Kingston, Kingston (CA); Kingston Health Sciences Centre, Kingston (CA)

(72) Inventors: David M. Berman, Kingston (CA); Palak Patel, Brantford (CA); John B.A. Okello, Toronto (CA); Robert Gooding, Kingston (CA); Atsunari Kawashima, Osaka (JP)

(21) Appl. No.: 17/281,809

(22) PCT Filed: Oct. 1, 2019
(86) PCT No.: PCT/CA2019/051403

§ 371 Date: Mar. 31, 2021

Related U.S. Application Data
(60) Provisional application No. 62/739,602, filed on Oct. 1, 2018.

(51) Int. Cl. C12Q 1/6886; C12Q 1/6858

(52) U.S. Cl. CPC C12Q 1/6886; C12Q 2600/118; C12Q 2600/154; C12Q 1/6858

(57) ABSTRACT

A multi-gene model is employed in methods for accurately classifying benign and malignant prostate cancer and reliably identifying prostate cancer in samples, with false positive and negative rates below 7%. A single gene model is employed in methods for detecting aggressive prostate cancer, prostate cancer patients at risk of developing biochemical recurrence, and prostate cancer patients suitable for treatment with an additional and/or alternative therapy. The methods may be implemented with next generation sequencing (NGS) or methylation-specific PCR (MSP). The MSP may use a mastermix specifically designed for use with bisulfite converted DNA in singleplex and multiplex assays.

### FIELD

The invention is in the field of prostate cancer detection using multi-gene models for classification of benign and malignant prostate with low false positive and false negative rates.

### BACKGROUND

High prevalence and low risk for progression have complicated efforts to screen and manage prostate cancer (PC). Serum prostate-specific antigen (PSA) testing is the most commonly used tool to identify men suspected of harboring PC. Patients with elevated PSA levels are typically referred for biopsy testing for definitive diagnosis. With a false positive rate of >75% and positive predictive value of ˜25%, PSA results are most often inconclusive. As a result, in the United States over 975,000 prostate biopsies were performed unnecessarily, leading to complications such as infection and bleeding and thousands of hospitalizations (Aubry et al., 2013).

The use of changes in mRNA and protein levels or genetic mutations for detection of PC has been investigated. However, mutations are very rare in PC (Tokheim et al., 2016), and only a handful of biomarkers (PSA, PCA3, TMPRSS2-ERG gene fusions) are currently utilized in tissue and urine/blood based diagnostic tests in clinics. In addition, such tests exhibit low balanced accuracy, with false positive or false negative rates of greater than 36%. In contrast, cancer-specific DNA methylation alterations are highly prevalent in PC, making them attractive targets. Only one DNA methylation test (ConfirmMDx; MDxHealth, Irvine, Calif.) has been marketed for PC, intended for men suspected of PC who have negative biopsies. However, like many other tests, this test suffers from low sensitivity and specificity (Partin et al., 2014; Stewart et al., 2013).

Epigenetic modification of DNA by methylation of cytosine residues has become a focus of research, with growing evidence supporting its role in progression and risk stratification of PC (Fraser et al., 2017; Ruggero et al., 2018; Vanaja et al., 2009). Therapeutic strategies based on methylation inhibitors have been proposed (Ngollo et al., 2014; Perry et al., 2010), while others are based on harnessing DNA methylation aberrations as useful diagnostic biomarkers (Valdés-Mora and Clark, 2015). To date, a majority of research in PC epigenetics has been discovery research, often using extensive, microarray-based screening to identify potential loci of interest. For example, it was found that GSTP1, APC, RASSF1A, PTGS2, and ABCB1 were hypermethylated in >85% of cancers (Yegnasubramanian et al., 2004). In another study, AOX1, CCDC181, GAS6, HAPLN3, KLF8, and MOB3B were added as cancer-specific methylation sites (Haldrup et al., 2013). Others expanded the search to find gene sets associated with recurrence or risk of progression (Lin et al., 2013; Mahapatra et al., 2012; Vanaja et al., 2009). In general, these studies have relied on vast sets of genes tested on comparatively low numbers of samples. Few have validated their findings in independent cohorts.

### SUMMARY

According to one aspect of the invention there is provided a mastermix for methylation-specific PCR (MSP), comprising: reaction buffer, 1×; deoxyribonucleotide triphosphate (dNTP), 50-500 μM; MgCl₂, 0-3.2 mM; DNA polymerase, 0.25 units (U); a concentration of BSA that stabilizes the DNA polymerase and neutralizes any potential inhibitors; and ROX reference dye, 24.5 nM.

In one embodiment, the mastermix is for use with genomic DNA, 50 ρg-1 μg.

In one embodiment, the mastermix is for use with bisulfite-converted genomic DNA, 50 ρg-1 μg.

In one embodiment, the mastermix is for a singleplex MSP, wherein: a gene forward primer concentration is 0.05-1 μM; a gene reverse primer concentration is 0.05-1 μM; and a gene probe or SYBR green dye concentration is 0.05-1 μM. In one embodiment, the gene forward primer concentration is 0.4 μM; the gene reverse primer concentration is 0.4 μM; and the gene probe or SYBR green dye concentration is 0.15 μM.

In one embodiment, the mastermix is for a multiplex MSP, wherein, for each gene: a gene forward primer concentration is 0.05-1 μM; a gene reverse primer concentration is 0.05-1 μM; and a gene probe concentration is 0.05-1 μM; wherein the multiplex MSP comprises 2, 3, or 4 genes. In one embodiment, for each gene: the gene forward primer concentration is 0.4 μM; the gene reverse primer concentration is 0.4 μM; and the gene probe concentration is 0.15 μM.

In one embodiment, a gene probe for a first gene is replaced with SYBR green dye.

According to another aspect of the invention there is provided an MSP method, comprising adding the following to a mastermix as described herein: bisulfite-converted DNA (50 ρg-1 μg), a gene forward primer (0.05-1 μM), a gene reverse primer (0.05-1 μM), and a gene probe or SYBR green dye (0.05-1 μM); mixing; performing PCR cycles including: heating to about 95° C. for about 30 seconds; about seven cycles of about 95° C. for about 30 seconds, cool to about 68° C. with about −2° C. touchdown for about 30 seconds, and hold at about 68° C. for about 30 seconds; about 48 cycles of about 95° C. for about 30 seconds, about 68° C. for about 30 seconds, and about 68° C. for about 30 seconds; and one cycle of about 68° C. for about five minutes.

In one embodiment, the MSP method is for multiplex MSP, wherein, for each gene: a gene forward primer concentration is 0.05-1 μM; a gene reverse primer concentration is 0.05-1 μM; and a gene probe concentration is 0.05-1 μM; wherein the multiplex MSP comprises 2, 3, or 4 genes. In one embodiment, a gene probe for a first gene is replaced with SYBR green dye.

According to another aspect of the invention there is provided a method for detecting prostate cancer in a subject, comprising: bisulfite converting genomic DNA obtained from the subject; mixing the bisulfite-converted DNA with a mastermix for amplifying and/or sequencing the DNA; wherein amplifying includes a selected region of each of GAS6, GSTP1, and HAPLN3 genes; detecting hypermethylation of the selected regions of the GAS6, GSTP1, and HAPLN3 genes; using the detected hypermethylation to identify prostate cancer in the subject.

In one embodiment, the method comprises using probes comprising SEQ ID NOs. 23, 8, and 35, or functional equivalents thereof, for the GAS6, GSTP1, and HAPLN3 genes, respectively. In one embodiment, the method comprises subjecting the detected hypermethylation of the GAS6, GSTP1, and HAPLN3 genes to a classifier to identify prostate cancer in the subject. In one embodiment, the selected hypermethylated region of the GAS6 gene is between a forward primer of SEQ ID NO: 22 and a reverse primer of SEQ ID NO: 24, or functional equivalents thereof; the selected hypermethylated region of the GSTP1 gene is between a forward primer of SEQ ID NO: 7 and a reverse primer of SEQ ID NO: 9, or functional equivalents thereof; and the selected hypermethylated region of the HAPLN3 gene is between a forward primer of SEQ ID NO: 34 and a reverse primer of SEQ ID NO: 36, or functional equivalents thereof.

The method may comprise amplifying using methylation-specific PCR (MSP).

The method may comprise amplifying and sequencing comprises using next generation sequencing (NGS).

The method may comprise mixing the bisulfite-converted DNA with a mastermix as described herein.

According to another aspect of the invention there is provided a method for detecting prostate cancer in a subject, comprising: bisulfate converting DNA obtained from the subject;

mixing the bisulfite-converted DNA with a mastermix for amplifying and/or sequencing the DNA; wherein amplifying includes a selected region of each of GSTP1, CCDC181, HAPLN3, GSTM2, GAS6, RASSF1, and APC genes; detecting hypermethylation of the selected regions of the GSTP1, CCDC181, HAPLN3, GSTM2, GAS6, RASSF1, and APC genes; using the detected hypermethylation to identify prostate cancer in the subject.

In one embodiment, the method comprises using probes comprising SEQ ID NOs. 8, 11, 35, 17, 23, 5, and 32, or functional equivalents thereof, for the GSTP1, CCDC181, HAPLN3, GSTM2, GAS6, RASSF1, and APC genes, respectively.

In one embodiment, the method comprises subjecting the detected hypermethylation of the GSTP1, CCDC181, HAPLN3, GSTM2, GAS6, RASSF1, and APC genes to a classifier to identify prostate cancer in the subject.

In one embodiment, the selected hypermethylated region of the GSTP1 gene is between a forward primer of SEQ ID NO: 7 and a reverse primer of SEQ ID NO: 9, or functional equivalents thereof; the selected hypermethylated region of the CCDC181 gene is between a forward primer of SEQ ID NO: 10 and a reverse primer of SEQ ID NO: 12, or functional equivalents thereof; the selected hypermethylated region of the HAPLN3 gene is between a forward primer of SEQ ID NO: 34 and a reverse primer of SEQ ID NO: 36, or functional equivalents thereof; the selected hypermethylated region of the GSTM2 gene is between a forward primer of SEQ ID NO: 16 and a reverse primer of SEQ ID NO: 18, or functional equivalents thereof; the selected hypermethylated region of the GAS6 gene is between a forward primer of SEQ ID NO: 22 and a reverse primer of SEQ ID NO: 24, or functional equivalents thereof; the selected hypermethylated region of the RASSF1 gene is between a forward primer of SEQ ID NO: 4 and a reverse primer of SEQ ID NO: 6, or functional equivalents thereof; and the selected hypermethylated region of the APC gene is between a forward primer of SEQ ID NO: 31 and a reverse primer of SEQ ID NO: 33, or functional equivalents thereof.

The method may comprise amplifying using methylation-specific PCR (MSP).

The method may comprise amplifying and sequencing comprises using next generation sequencing (NGS).

The method may comprise mixing the bisulfite-converted DNA with a mastermix as described herein.

According to another aspect of the invention there is provided method for identifying a prostate cancer patient at risk of developing biochemical recurrence, and/or suitable for treatment with an additional and/or alternative therapy, comprising: bisulfite converting genomic DNA obtained from the subject; mixing the bisulfite-converted DNA with a mastermix for amplifying and/or sequencing the DNA; wherein amplifying includes a selected region in UCHL1 gene; detecting hypermethylation of the selected region of the UCHL1 gene; using the detected hypermethylation to identify risk of developing biochemical recurrence of prostate cancer, and/or suitability for treatment with an additional and/or alternative therapy.

One embodiment comprises using a probe comprising SEQ ID NO. 44, or a functional equivalent thereof, for the UCHL1 gene, respectively.

In one embodiment the selected hypermethylated region of the UCHL1 gene is between a forward primer of SEQ ID NO: 43 and a reverse primer of SEQ ID NO: 45, or a functional equivalent thereof.

In one embodiment amplifying comprises using methylation-specific PCR (MSP).

In one embodiment the method comprises using a mastermix as described herein.

In one embodiment amplifying and sequencing comprises using next generation sequencing (NGS).

In the aspects and embodiments described herein, the genomic DNA may be obtained from a biological sample selected from fresh/frozen prostate tissue, archival prostate tissue including formalin fixed and paraffin embedded (FFPE tissue), blood, and urine.

According to another aspect of the invention there is provided a kit for detecting prostate cancer comprising a mastermix as described herein, primers and probes for a selected methylation site in each of GAS6, GSTP1, and HAPLN3 genes, and instructions for detecting prostate cancer.

According to another aspect of the invention there is provided a kit for detecting prostate cancer comprising a mastermix as described herein, primers and probes for a selected methylation site in each of GSTP1, CCDC181, HAPLN3, GSTM2, GAS6, RASSF1, and APC genes, and instructions for detecting prostate cancer.

According to another aspect of the invention there is provided a kit for detecting aggressive prostate cancer, prostate cancer patients at risk of developing biochemical recurrence, and/or prostate cancer patients suitable for treatment with an additional and/or alternative therapy, comprising a mastermix as described herein, primers and probes for a selected methylation site in USCHL1 gene, and instructions for use.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a volcano plot showing changes in DNA methylation levels between benign and cancer samples for 14/15 genes in the training dataset with fold change of >2, and corresponding adjusted p-values (after Bonferroni correction) from the Mann-Whitney U test.

FIGS. 2A-2C are box plots and ROC curves of the seven genes with the highest DNA methylation changes from the training dataset, wherein distribution of the normalized methylation levels in cancer (right boxes) and benign (left boxes) samples for each DNA methylation change is shown with corresponding ROC curve.

FIG. 3A is an ROC curve showing performance of a three-gene classifier (GAS6/GSTP1/HAPLN3) in the training dataset; also shown is the AUC and model threshold of 0.917.

FIG. 3B is a plot showing performance of the three-gene binary classifier tested on the validation dataset, where the horizontal line at 0.917 shows the model threshold from FIG. 3A.

FIG. 4 is a plot showing changes in DNA methylation levels at GSTP1 and GAS6 loci in a urine sample, using methylation specific PCR (MSP).

FIG. 5 is a plot showing percent methylation at selected CpG sites for various loci in a urine sample, using next generation sequencing (NGS).

FIGS. 6A and 6B are Kaplan-Meier survival curve analyses demonstrating higher risk of biochemical recurrence (BCR) in patients with hypermethylation at UCHL1 (A and B) locus, in both training and validation cohorts.

FIGS. 7A and 7B are typical real-time PCR amplification plots of singleplex and multiplex reactions, respectively, using a mastermix formulation, according to embodiments of the invention, wherein the APC amplification curve is represented as solid black lines in both graphs.

FIG. 8 is a plot showing a standard curve of an APC MSP assay wherein four-fold serial dilution of bisulfite-treated DNA was performed, and the assay was carried out in multiplex setting; cycle threshold (Cq) values corresponding to each dilution point are plotted on the y-axis and associated statistics are shown below the graph.

FIG. 9 is a plot showing a comparison of a mastermix according to one embodiment (solid lines) with a commercially available PCR mix (dashed lines) performed by assessing differences in their respective amplification curves.

### DETAILED DESCRIPTION OF EMBODIMENTS

As described herein, 15 genes (Table 1) that are frequently methylated in PC were selected for quantitative DNA methylation analysis (one region was selected for each gene). Due to lack of thorough validation or limited sample sizes, clinical utility of these genes or regions in prostate cancer detection has not been fully demonstrated previously. Methylation-specific PCR (MSP) assays were employed to measure methylation levels in the selected regions in over 1250 cancer and ˜95 benign radical prostatectomy (RP) samples (from 699 RP cases) divided into independent training and validation cohorts. Using this data, seven of the gene regions were identified as being useful candidates for accurately identifying prostate cancer in the samples.

TABLE 1
Ensembl ID numbers and assay locations of
the 15 genes used in the embodiments.

| Gene | Ensembl_ID | Assay location (GRCh38) |
|---|---|---|
| ABCB1 | ENSG00000085563 | Chromosome 7: 87,600,293-87,600,383 |
| ALDH1A2 | ENSG00000128918 | Chromosome 15: 58,065,234-58,065,328 |
| AOX1 | ENSG00000138356 | Chromosome 2: 200,586,275-200,586,363 |
| APC | ENSG00000134982 | Chromosome 5: 112,737,742-112,737,819 |
| CCDC181 | ENSG00000117477 | Chromosome 1: 169,427,513-169,427,622 |
| GAS6 | ENSG00000183087 | Chromosome 13: 113,862,976-113,863,066 |
| GSTM2 | ENSG00000213366 | Chromosome 1: 109,668,034-109,668,161 |
| GSTP1 | ENSG00000084207 | Chromosome 11: 67,583,508-67,583,612 |
| HAPLN3 | ENSG00000140511 | Chromosome 15: 88,895,312-88,895,412 |
| HIC1-M | ENSG00000177374 | Chromosome 17: 2,056,616-2,056,716 |
| HOXD3 | ENSG00000128652 | Chromosome 2: 176,159,941-176,160,042 |
| PTGS2 | ENSG00000073756 | Chromosome 1: 186,680,681-186,680,756 |
| RASSF1A | ENSG00000068028 | Chromosome 3: 50,340,817-50,340,892 |
| SEPT9 | ENSG00000184640 | Chromosome 17: 77,373,470-77,373,557 |
| UCHL1 | ENSG00000154277 | Chromosome 4: 41,256,742-41,256,831 |

From that data a highly sensitive and specific three-gene classifier for PC was constructed and validated. In one embodiment the classifier is based on a statistical model that utilizes the changes in levels of DNA methylation in the selected regions of GAS6, GSTP1, and HAPLN3 genes to accurately identify malignant prostate tissue samples. The model can identify samples exhibiting prostate cancer using DNA methylation levels of these three genes with accuracy of about 99%. Thorough validation of the classifier in over one thousand samples from an independent patient population has confirmed the utility and clinical feasibility of the model.

Embodiments may employ methods other than MSP for DNA methylation analysis, such as, for example, next generation sequencing (NGS).

### Patient Material

As part of a larger genomic profiling study, three patient cohorts were analyzed. They consisted of consecutive radical prostatectomies performed with curative intent for histologically verified clinically localized PC (Table 2). Cohorts were obtained from Kingston General Hospital (KGH; Kingston, Ontario) (2000-2012), McGill University/Montreal General Hospital (MGH; Montreal, Quebec) (1994-2013) and London Health Science Centre (LHSC; London, Ontario) (2003-2009). In total, 699 patients were included in the study.

TABLE 2
Clinicopathologic characteristics of radical prostatectomy
patients included in this study.

| | Training | | | | | | Validation | |
|---|---|---|---|---|---|---|---|---|
| | KGH cohort | | MGH cohort | | Total | | LHSC cohort | |
| Total Cases | 223 | | 257 | | 480 | | 219 | |
| Benign | 17 | (7.6%) | 24 | (9.3%) | 41 | (8.5%) | 55 | (25.1%) |
| Cancer | 223 | (100%) | 257 | (100%) | 480 | (100%) | 219 | (100%) |
| Grade Group | | | | | | | | |
| 1 | 45 | (20.2%) | 37 | (14.4%) | 82 | (17.1%) | 78 | (35.6%) |
| 2 | 149 | (66.8%) | 151 | (58.8%) | 300 | (62.5%) | 132 | (60.3%) |
| ≥3 | 29 | (13%) | 69 | (26.8%) | 98 | (20.4%) | 9 | (4.1%) |
| Stage | | | | | | | | |
| T2 | 171 | (76.7%) | 140 | (54.4%) | 311 | (64.8%) | 177 | (80.8%) |
| T3a | 44 | (19.7%) | 102 | (39.7%) | 146 | (30.4%) | 36 | (16.4%) |
| T3b | 8 | (3.6%) | 15 | (5.8%) | 23 | (4.8%) | 6 | (2.7%) |

A previously published protocol (Patel et al., 2016) was used to macro-dissect and extract DNA from index tumour foci from 699 RP cases and benign regions from RP cases, yielding over 1300 tissue samples (formalin fixed and paraffin embedded; FFPE). DNA was quantified on a Qubit 3.0 Fluorometer (Thermo Fisher Scientific) using the dsDNA HS (High Sensitivity) kit. A summary of final sample numbers for each DNA methylation assay is shown in Table 3.

TABLE 3
Summary of samples and patients for each DNA methylation loci investigated.

| | Training (Queen's + McGill cohort) | | | | Validation (LHSC cohort) | | | |
|---|---|---|---|---|---|---|---|---|
| | Cases: | Cases: | Samples: | Samples: | Cases: | Cases: | Samples: | Samples: |
| Genes | Benign | Cancer | Benign | Cancer | Benign | Cancer | Benign | Cancer |
| ABCB1 | 39 | 470 | 39 | 871 | 52 | 212 | 52 | 364 |
| ALDH1A2 | 40 | 469 | 40 | 874 | 52 | 214 | 52 | 364 |
| AOX1 | 36 | 428 | 36 | 749 | 31 | 129 | 31 | 196 |
| APC | 41 | 473 | 41 | 869 | 55 | 218 | 55 | 377 |
| CCDC181 | 40 | 470 | 40 | 872 | 51 | 206 | 51 | 352 |
| GAS6 | 36 | 431 | 36 | 780 | 30 | 137 | 30 | 213 |
| GSTM2 | 38 | 465 | 38 | 864 | 52 | 213 | 52 | 361 |
| GSTP1 | 41 | 476 | 41 | 888 | 55 | 218 | 55 | 376 |
| HAPLN3 | 41 | 474 | 41 | 872 | 52 | 214 | 52 | 361 |
| HIC1-M | 41 | 477 | 41 | 890 | 55 | 218 | 55 | 375 |
| HOXD3 | 39 | 467 | 39 | 853 | 40 | 171 | 40 | 277 |
| PTGS2 | 41 | 474 | 41 | 875 | 55 | 215 | 55 | 363 |
| RASSF1A | 41 | 475 | 41 | 867 | 55 | 216 | 55 | 368 |
| SEPT9 | 41 | 474 | 41 | 868 | 55 | 218 | 55 | 375 |
| UCHL1 | 41 | 477 | 41 | 890 | 55 | 215 | 55 | 371 |

### Methylation-Specific PCR (MSP) Analysis

Real time MSP assays were performed as previously described (Olkhov-Mitsel et al. 2014; Patel et al. 2016, 2017) targeting the selected methylation regions on the 15 genes (Table 4) in DNA samples collected from three RP cohorts. Briefly, individual DNA samples (50 ng) were bisulfite converted according to the manufacturer's protocol (EpiTect Bisulfite Kit, Qiagen). A mastermix was developed for this assay, embodiments of which are described below. In one embodiment used in this analysis, the mastermix included one of 15 primer pairs (400 nM; Thermo Fisher Scientific) and probe sets (150 nM; Thermo Fisher Scientific) (Table 4), nucleotides (250 μM; Invitrogen), MgCl₂ (1.2 mM; NEB), BSA (0.5 mg/mL; NEB), ROX reference dye (24.5 nM; Invitrogen), EpiMark Taq polymerase (0.25 U; NEB) and 1× EpiMark reaction buffer (NEB) was prepared. Next, bisulfite-converted DNA (1 μL) was added to the mastermix and MSP reactions (10 uL) were carried out in a VIIA7 thermocycler (Applied Biosystems). The cycling conditions included denaturation at 95° C. for 30 s, 7 cycles of touchdown PCR with annealing temperatures decreasing by 2° C. per cycle and extension at 68° C. for 30 s, followed by 48 cycles of 30 s at 95° C., 30 s at 58° C., 30 s at 68° C., and a final extension step of 5 min at 68° C.

An assay targeting Alu repeat elements was used as the reference control and distilled water was used as a negative control. CpG methylated Jurkat DNA (New England Biolabs) was used as a positive control sample, and assay efficiency of each MSP assay was determined by generating standard curves as described previously (Bustin, et al. 2009) (Table 4).

TABLE 4
Primer pair and probe sequences and amplification efficiencies of each MSP assay
(F = forward; P = probe; R = reverse).

| | | SEQ | | | | |
|---|---|---|---|---|---|---|
| | | ID | Assay | Efficiency | Amplification | |
| Assay | Sequence | NO. | Type | (%) | factor | Description |
| ABCB1 | F: AAACGCCCGCCGTTAATA | 1 | Target | 91.53 | 1.92 | ATP-binding |
| | P: CCCAACTACTCTAACCGCGATAAACACT | 2 | | | | cassette, sub- |
| | R: TTCGTGGAGATGTTGGAGATTT | 3 | | | | family B |
| | | | | | | (MDR/TAP), |
| | | | | | | member 1 |
| RASSF1A | F: GCGTTGAAGTCGGGGTTC | 4 | Target | 94.77 | 1.95 | Ras association |
| | P: ACAAACGCGAACCGAACGAAACCA | 5 | | | | (RalGDS/AF-6) |
| | R: CCCGTACTTCGCTAACTTTAAACG | 6 | | | | domain family |
| | | | | | | member 1 |
| GSTP1 | F: ATGTTTCGGCGCGTTAGT | 7 | Target | 88.68 | 1.89 | Glutathione S- |
| | P: TCGTTGCGTATATTTCGTTGCGGT | 8 | | | | transferase pi 1 |
| | R: ACCTTTCCCTCTTTCCCAAATC | 9 | | | | |
| CCDC181 | F: ATCGATTTTTTTCGGTATTTA | 10 | Target | 81.05 | 1.81 | Coiled-coil |
| | P: TCGGTGTTTGCGAAGGGTTAG | 11 | | | | domain |
| | R: GCGAATCTCTATAACGTTAATAA | 12 | | | | containing 181 |
| Alu | F: GCGGTGGTTTACGTTTGTAAT | 13 | Control | 89.2 | 1.89 | Repeat element |
| | P: AAATAATCCGCCCGCCTCGACC | 14 | | | | |
| | R: AAATAATCTCGATCTCCTAACCTCA | 15 | | | | |
| ALDH1A2 | F: GGATCGGGAGAATCGGATAGAA | 16 | Target | 92.21 | 1.92 | Aldehyde |
| | P: AAACAACCGTCAACGACTCCTACCC | 17 | | | | dehydrogenase 1 |
| | R: ACTTAACCCAACCCGAAACG | 18 | | | | family, member |
| | | | | | | A2 |
| GSTM2 | F: CTCTACCCTCTCTAAACCTCTCA | 19 | Target | 91.82 | 1.92 | Glutathione S- |
| | P: TGGGTATGGTGTTGGTTGTTGTGGA | 20 | | | | transferase mu 2 |
| | R: TTTGGTTTATTTCGCGGATGTT | 21 | | | | (muscle) |
| GAS6 | F: AACATTCCTAACCGAAATACCG | 22 | Target | 91.22 | 1.91 | Growth arrest- |
| | P: ACACCGTCGAACTCCTAA | 23 | | | | specific 6 |
| | R: CGGTTTCGTTTTGTTAGGTG | 24 | | | | |
| SEPT9 | F: CGCGCGATTCGTTGTTTATTAG | 25 | Target | 97 | 1.97 | Septin 9 |
| | P: CGGTTAACGCGTAGTTGGATGGGA | 26 | | | | |
| | R: CCCACCTTCGAAATCCGAAATA | 27 | | | | |
| HIC1 | F: GTTAGGCGGTTAGGGCG | 28 | Control | 102.91 | 2.03 | Hypermethylated |
| | P: CGTAGGAGAGTGTGTTGGGTAGAC | 29 | | | | in cancer 1 |
| | R: CCGAACGCCTCCATCG | 30 | | | | |
| APC | F: GGAAGCGGAGAGAGAAGTAG | 31 | Target | 85.49 | 1.85 | Adenomatous |
| | P: AATTCGTTGGATGCGGATTAG | 32 | | | | polyposis coli |
| | R: GACGAACTCCCGACGAAA | 33 | | | | |
| HAPLN3 | F: GTTTTCGTAGTGTTCGGTTTAC | 34 | Target | 98.8 | 1.99 | Hyaluronan and |
| | P: TCGGATTTTGTTCGGGAGGT | 35 | | | | proteoglycan link |
| | R: GAATTCCTCCCTTACCGC | 36 | | | | protein 3 |
| AOX1 | F: TTTTTTTCGTAATAGCGGTTTTGT | 37 | Target | 97.84 | 1.98 | Aldehyde oxidase |
| | P: TCGTATTTTTATTTTTGTTTTCGGG | 38 | | | | 1 |
| | R: ATCCAAAACAATCCCTAAAAACG | 39 | | | | |
| HOXD3 | F: GGGTGTTTAGGGATTAGAGAGG | 40 | Target | 90.8 | 1.91 | Homeobox D3 |
| | P: TTTGGGTTCGGGTCGTTTGTTACG | 41 | | | | |
| | R: CGAACTCAACAACCGAATCAC | 42 | | | | |
| UCHL1 | F: CGGCGAGTGAGATTGTAAGGTT | 43 | Target | 96.49 | 1.96 | Ubiquitin |
| | P: TTCGGTCGTATTATTTCGCGTTGCGTAC | 44 | | | | Carboxyl-Terminal |
| | R: GAACGATCGCGACCAAATAAATA | 45 | | | | Esterase L1 |
| | | | | | | (Ubiquitin |
| | | | | | | Thiolesterase) |
| PTGS2 | F: AATTCCACCGCCCCAAAC | 46 | Target | 103.45 | 2.03 | Prostaglandin- |
| | P: ATTTGGCGGAAATTTGTGC | 47 | | | | Endoperoxide |
| | R: CGGAAGCGTTCGGGTAAAG | 48 | | | | Synthase 2 |
| | | | | | | (Prostaglandin |
| | | | | | | G/H Synthase and |
| | | | | | | Cyclooxygenase) |

### Data Analysis and Statistics

The relative threshold method, Cᵣₜ (Applied Biosystems Relative Quantification (“RQ”) application on ThermoFisher Cloud) was used to determine cycle quantification (C_{q}) values for each amplification curve. Cᵣₜ parameter optimization (Early access version, ThermoFisher Scientific Cloud) was conducted to enhance reliable detection of amplification. Sample reactions with inconclusive amplification curves, contamination, or poor reaction efficiency were excluded from further analysis. Reactions with confirmed negative amplification were assigned two C_{q} values higher than the maximum observed C_{q} value in the respective cohort. Number of samples included in downstream analysis are listed in Table 3. Normalized methylation levels or abundance ratios were calculated using delta-delta Cₜ method (Pfaffl, 2001) as described below:

$$Normalized methylation levels = \frac{{2}^{( {P}_{t} - {S}_{t} )}}{{2}^{( {P}_{r} - {S}_{r} )}}$$

Where,
Pₜ=C_{q} of positive control DNA control for target gene;
Sₜ=C_{q} of sample for target gene;
Pᵣ=C_{q} of positive control DNA for reference (Alu);
Sᵣ=C_{q} of sample for reference (Alu)

Exploratory analyses were performed using the training cohort dataset, and differential methylation levels of the 15 selected DNA regions were assessed as fold changes using a Mann-Whitney test. p values were adjusted for false discovery using the family-wise Bonferroni method. All DNA methylation changes with significant enrichment in cancer samples compared to benign were considered for downstream analysis. After testing several supervised learning algorithms including liner regression, linear and quadratic discriminant analysis, and support vector machines, logistic regression was identified as the most suitable for the analyses as it consistently produced better classifiers. Univariate and multivariate logistic regression analysis assessing all possible combinations of DNA methylation changes were performed and the resulting models were ranked according to their balanced accuracy. Receiver operating characteristic (ROC) curve analysis, areas under these curves (AUC), and confusion matrices were generated for best-performing models using model thresholds determined from the “closest topleft” method (R Core Team, 2017; Robin et al., 2011). The best model was selected using the training cohort dataset, and was then applied to the validation cohort dataset. Statistical analysis was performed in R (v3.4.1) using “pROC”, “caret”, “ggrepel” and “ggplot2” packages (Kamil Slowikowski, 2017; R Core Team, 2017; Robin et al., 2011; Wickham, 2009).

### Common Methylation Changes in Prostate Cancer

The three radical prostatectomy cohorts from which over 1300 DNA samples extracted (see Tables 2 and 3) were selected originally to study prognostic biomarkers. However, as a by-product of that work, diagnostic biomarkers were identified using the following approach. Cases from two cohorts with 41 benign samples and 890 cancer samples from 480 patients were merged into a training dataset. An independent cohort from a 3rd hospital (LHSC) contained 55 benign samples and 377 cancer samples from 219 patients, and was used for validation.

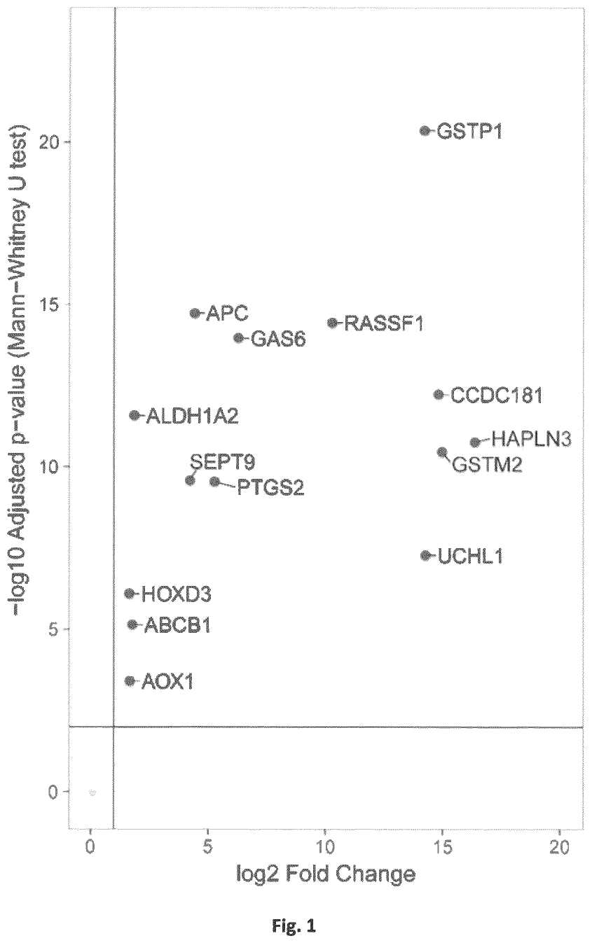

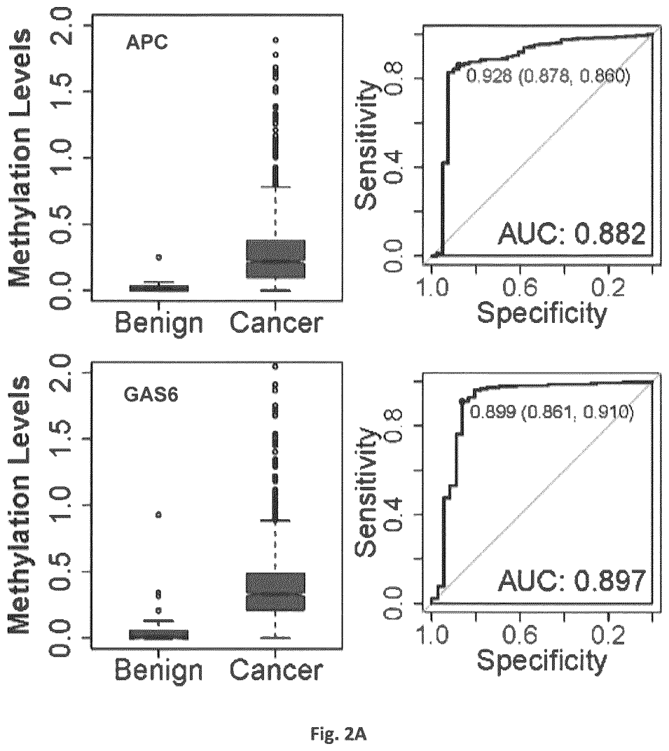

Real-time MSP assays were used to profile methylation changes in small (˜100 bp) regions covering 15 CpG islands which are frequently hypermethylated in PC. In the training dataset, 14 out of 15 of these regions were significantly hypermethylated (adjusted P value <0.01) with normalized methylation levels or abundance ratios >2) in 890 cancer samples compared to 41 benign samples (FIG. 1). In contrast, methylation levels of the HIC1 CpG island were similar in cancer and benign samples, possibly representing a cancerization field effect (Yegnasubramanian et al., 2004). In particular, seven DNA methylation changes at GSTP1, CCDC181, HAPLN3, GSTM2, GAS6, RASSF1, and APC showed the largest differences between cancer and benign (FIGS. 2A-2C). For each of these seven regions, DNA methylation levels in benign samples were minimal with low variation (FIGS. 2A-2C). In a univariate logistic modelling of the training dataset, the area under the curve from ROC analysis for each of these regions ranged from 83% to 95%, individually. The specificity of these univariate logistic models ranged from 77% to 90%, and the sensitivity ranged from 72% to 91% (FIGS. 2A-2C). The area under the curve for each of the ROC curves is annotated with sensitivity and specificity corresponding to the best threshold (according to the “closest.topleft” method).

GSTP1 was highly methylated (i.e., hypermethylated) in cancer, but not in benign samples. As a cancer classifier, GSTP1 alone demonstrated an AUC of 95% and balanced accuracy of 88%. TCGA PC data show similar results (The Cancer Genome Atlas Research Network, 2015). Two other loci, GAS6 and APC, demonstrated strong diagnostic capabilities with comparable balanced accuracies to GSTP1, but with AUCs of <90%. It was found that regardless of the model threshold chosen, each single gene had false positive and/or false negative rates of 10% or higher. Therefore, to improve accuracy multigene logistic modelling was performed.

### Multigene Diagnostic Model in Prostate Cancer

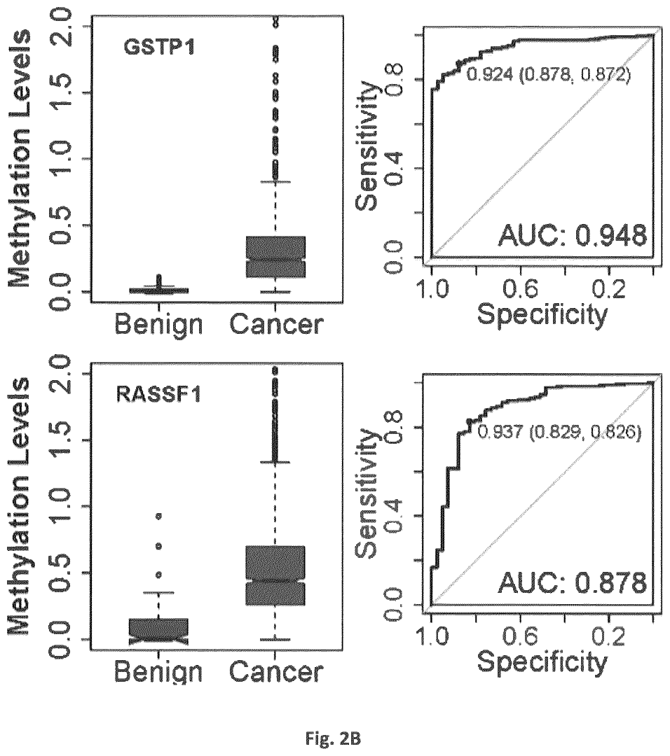

The multivariate approach chosen relied on the simplest binary classifier model, logistic regression. Using the training dataset, all possible combinations of all 15 methylation regions were tested to identify a multigene model with higher sensitivity and specificity. A three-gene model based on GAS6/GSTP1/HAPLN3 was selected as the best binary (i.e., cancer/benign) classifier with an AUC of 97% for the ROC curve (Table 5, FIG. 3A). Using the closest top-left method, the threshold was determined to be 0.917 for the three-gene model, which produced specificity and sensitivity of 92% (FIG. 3A). A summary of the performance of one, two, or three gene models using GAS6/GSTP1/HAPLN3 DNA methylation is shown in Table 6.

TABLE 5
Summary of the thee-gene classifier function (logistic
regression) developed using the training dataset.

| Reporter | Coefficient | Std. Error | p-value |
|---|---|---|---|
| GSTP1 | 48.99 | 9.24 | 1.17E−07 |
| GAS6 | 10.48 | 2.03 | 2.45E−07 |
| HAPLN3 | −9.05 | 1.70 | 9.66E−08 |

TABLE 6
Comparison of performance characteristics of models based
on GSTP1, GAS6 and HAPLN3 in the training dataset.

| | Total | | | | | | | Balanced | |
|---|---|---|---|---|---|---|---|---|---|
| Gene | samples | TN | FP | FN | TP | Sensitivity | Specificity | Accuracy | AUC |
| GAS6 | 816 | 31 | 5 | 70 | 710 | 0.910 | 0.861 | 0.886 | 0.897 |
| GSTP1 | 929 | 36 | 5 | 114 | 774 | 0.872 | 0.878 | 0.875 | 0.948 |
| HAPLN3 | 913 | 33 | 8 | 151 | 721 | 0.827 | 0.805 | 0.816 | 0.828 |
| GAS6 + GSTP1 | 812 | 32 | 4 | 102 | 674 | 0.869 | 0.889 | 0.879 | 0.953 |
| GAS6 + HAPLN3 | 811 | 31 | 5 | 65 | 710 | 0.916 | 0.861 | 0.889 | 0.896 |
| GSTP1 + HAPLN3 | 911 | 37 | 4 | 127 | 743 | 0.854 | 0.902 | 0.878 | 0.942 |
| GAS6 + GSTP1 + HAPLN3 | 810 | 33 | 3 | 64 | 710 | 0.917 | 0.917 | 0.917 | 0.972 |

Having optimized an accurate binary classifier, the same threshold was used to validate the GAS6/GSTP1/HAPLN3 model in an independent cohort. As shown in Table 7 and FIG. 3B, the three-gene model (GAS6/GSTP1/HAPLN3), misclassified only 2/30 benign samples (6.7%) from the validation dataset as cancer. As for the cancer samples, only 12 out of 212 samples (5.6%) were misclassified as benign. In FIG. 3B, the horizontal line at 0.917 shows the model threshold. The three-gene model showed sensitivity of 94% and specificity of 93% in the validation dataset. Overall, the GAS6/GSTP1/HAPLN3 model showed a significant improvement over univariate approaches, with a balanced accuracy of 94%, positive predictive value (PPV) of 99% and a negative predictive value (NPV) of 70% in the validation dataset (Table 7).

TABLE 7
Confusion matrix and associated statistics showing performance
of the GAS6/GSTP1/HAPLN3 model in the validation dataset.
Confusion Matrix

Actual

| | | Benign | Cancer |
|---|---|---|---|
| Prediction | Benign | 28 | 12 |
| | Cancer | 2 | 200 |

| | |
|---|---|
| Specificity | 0.93 |
| Pos Pred Value | 0.99 |
| Neg Pred Value | 0.70 |
| Balanced Accuracy | 0.94 |

The embodiments described herein provide and validate differentially and consistently hypermethylated genomic loci in PC, along with inexpensive assays that are expected to be compatible with routine workflow in clinical laboratories. The superior performance of the three-gene classifier (GAS6/GSTP1/HAPLN3) demonstrated in tissue samples as described herein provides compelling evidence suggesting the classifier's use in other non-invasive assays, such as urine or blood tests.

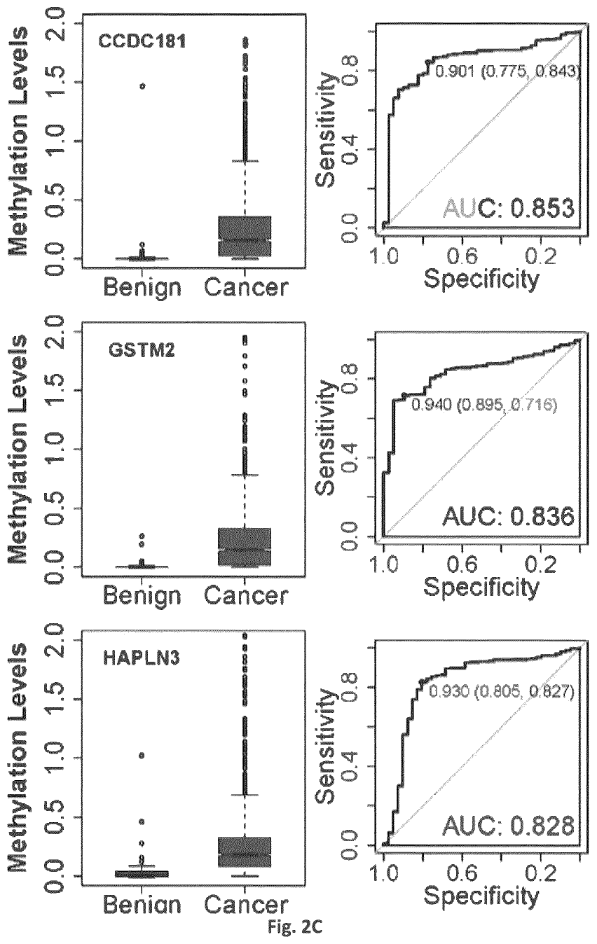

For example, to demonstrate use with urine, urine was collected from a patient with early stage prostate cancer after attentive digital rectal examination. DNA was isolated from 5 mL of the urine using a Urine DNA Isolation Kit—Slurry Format (Norgen Biotek Corp., Thorold, ON, Canada). The DNA (25 ng) was bisulfite converted using EpiTect® Bisulfite Conversion Kit (Qiagen, Toronto, ON, Canada). Bisulfite converted DNA was used in quantitative methylation specific PCR (MSP). As shown in FIG. 4, DNA methylation changes at GSTP1 and GAS6 promoter regions were reliably detected in 5 mL of urine sample.

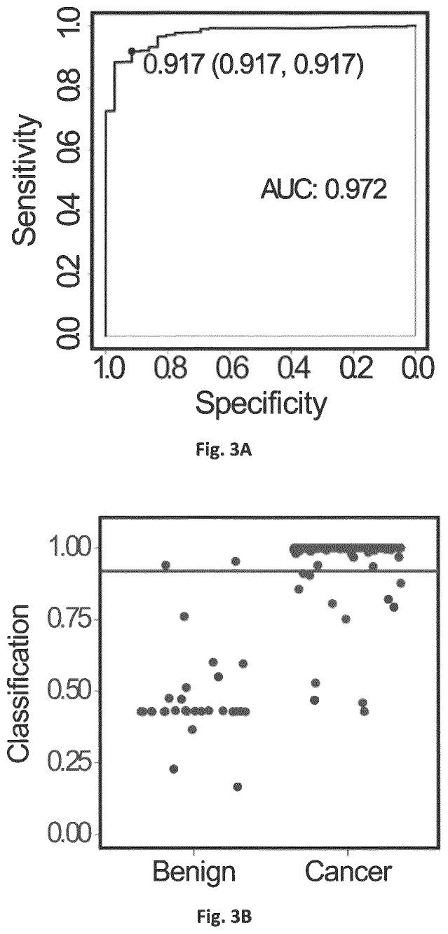

In addition, a urine sample was subject to next generation sequencing (NGS). DNA was isolated from 5 mL of urine collected from a patient with early stage prostate cancer after attentive digital rectal examination. The DNA (50 ng) was bisulfite converted using the MethylCode™ Bisulfite Conversion Kit (Thermo Fisher Scientific Inc.). An AmpliSeg™ (Illumina, Inc.) multiplex library construction protocol was performed, followed by automated templating with the Ion 520 & Ion 530 ExT Kit-Chef™ (Thermo Fisher Scientific Inc.) and Ion S5 Sequencing. Analysis was performed using the Methylation Analysis plugin available for the Torrent™ Suite Software (Thermo Fisher Scientific Inc.). FIG. 5 shows DNA methylation (%) measured by massive parallel sequencing (Thermo Fisher Scientific Inc.) at selected CpG sites for each locus listed on the x-axis. It can be seen that methylation of GAS6, GSTP1, and HAPLN3 was detected.

Association of DNA Methylation Alterations with Biochemical Recurrence in Prostate Cancer

While cancer specific survival (CSS) and overall survival (OS) are most often used to describe prognosis in cancer, prostate cancer is diagnosed early and progresses very slowly, with few men dying, and most deaths occurring after ˜20 years of diagnosis. Thus, cancer recurrence after surgery or radiotherapy has been adopted as a more practical surrogate of mortality-related indices. Recurrence, usually detected by rising PSA levels and therefore referred to as biochemical recurrence (BCR), has been associated with metastatic disease progression and prostate cancer-specific mortality. After prostatectomy, the presence of BCR typically pre-dates the appearance of metastasis by about eight years, and prostate cancer-specific mortality by about 15 years. As a result, BCR is widely used to assess treatment success and manage secondary therapy decisions. Unfortunately, defining BCR after treatment of localized prostate cancer is challenging because the post-treatment PSA level which is indicative of prostate cancer recurrence varies with the type of therapy. Many BCR definitions have been proposed in the literature for patients who have undergone radical prostatectomy (e.g., Stephenson et al., 2006; Cookson et al., 2007; Amling et al., 2001), each of them associated with varying probability of prostate cancer progression. After consulting with lead urologists in Canada, the American Urological Association Prostate Guideline Update Panel's recommended BCR definition was used for this study, and patients with two consecutive PSA values of >0.2 ng/mL after prostatectomy were identified as recurrent.

The number of cases with BCR in the Queen's, McGill, and LHSC cohorts were 51 (22.9%), 52 (20.2%), and 19 (8.7%), respectively. In contrast to analyses involving prostate cancer grade group as an endpoint, to study BCR in prostate cancer, samples from McGill and LHSC cohorts were combined to form a training dataset (which included 71 BCR patients) as the proportion of cases with BCR was higher in the Queen's cohort. The Queen's cohort (n=399 samples from n=223 cases) was used for validation, since it had the highest fraction of recurrent cases. Cases form McGill and LHSC cohorts were combined to form a training cohort (n=879 samples from n=475 cases).

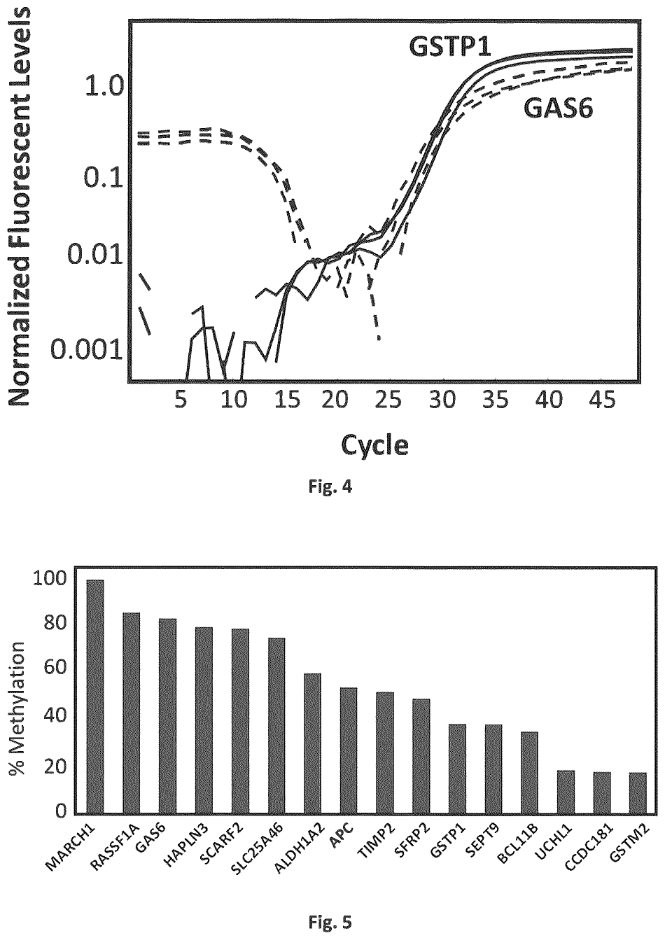

Using log-rank statistics, we selected cut-offs that maximize association of DNA methylation alterations with BCR-free survival, and dichotomized DNA methylation changes at each of the 15 loci (Table 4). DNA hypermethylation at UCHL1 locus was found to be an independent risk factor (hazard ratio >2.25) for BCR in the training and validation cohorts (Table 8). Kaplan-Meier curve analysis further verified these findings and showed that patients with hypermethylation at UCHL1 locus experience BCR at a significantly faster pace in both training and validation cohorts (FIGS. 6A and 6B, respectively). The rest of the DNA methylation alterations were not significant in multivariate analysis. The results confirm the use of UCHL1 hypermethylation for identifying patients with higher risk of developing biochemical recurrence. The results also indicate that detecting UCHL1 hypermethylation may be useful in a variety of postsurgical settings for identifying patients with aggressive prostate cancer. The results also indicate that detecting UCHL1 hypermethylation may be useful for identifying patients who may benefit from additional and/or alternative therapies, such as adjuvant radiation therapy.

TABLE 8
Multivariate Cox's proportional hazards model for BCR-free survival in association
with DNA hypermethylation at UCHL1 locus (HR = hazard ratio, CI = confidence
interval, GG = prostatectomy grade group, SVI = seminal vesicle invasion, EPE =
extraprostatic extension, and SM = surgical margin).

| | Training cohort | | Validation cohort | |
|---|---|---|---|---|
| Variable | HR (95% CI) | p-value | HR (95% CI) | p-value |
| UCHL1 | | | | |
| High vs Low | 2.27 (1.09-4.72) | 0.029 | 2.35 (1.05-4.75) | 0.036 |
| Prostatectomy Grade Groups | | | | |
| GG2 vs GG1 | 1.80 (0.61-5.25) | 0.29 | 2.32 (0.79-6.83) | 0.12 |
| ≥GG3 vs GG1 | 2.57 (0.82-8) | 0.1 | 2.83 (0.80-9.99) | 0.11 |
| Age | | | | |
| 1 unit increase | 1.01 (0.96-1.05) | 0.8 | 1.04 (0.98-1.10) | 0.16 |
| Pre-operative PSA, ng/ml | | | | |
| 6-10 vs <6 | 1.41 (0.78-2.53) | 0.25 | 0.69 (0.36-1.33) | 0.27 |
| 11-20 vs <6 | 2.74 (1.34-5.60) | 0.006 | 1.55 (0.70-3.44) | 0.28 |
| >20 vs <6 | 1.98 (0.64-6.19) | 0.24 | 4.96 (0-inf) | 0.99 |
| SVI | | | | |
| Yes vs No | 2.32 (0.90-5.94) | 0.08 | 2.43 (0.92-6.44) | 0.07 |
| EPE | | | | |
| Yes vs No | 1.47 (0.85-2.55) | 0.17 | 1.50 (0.74-3.03) | 0.26 |
| SM | | | | |
| Yes vs No | 2.30(1.35-3.94) | 0.002 | 2.22 (1.09-4.49) | 0.027 |

To obtain the data, patients with two consecutive post-surgery PSA levels above 0.2 were considered biochemical recurrence events. As multiple samples were collected from each prostatectomy patient, the highest normalized methylation levels were used to tabulate patient-based dataset for each of 15 DNA methylation alterations. The patient-based datasets, from training and validation cohorts, were used in the downstream analyses. Using the MaxStat™ package (MaxStat Software, Jever-OT Cleverns, Germany), an outcome-oriented method was used to select a cut-off point for each DNA methylation alteration in the training cohort. The cut-off corresponded to the most significant association with BCR-free survival. The cut-offs selected from the training cohort were validated in an independent cohort. Kaplan-Meier estimates of BCR-free survival were graphically plotted for significant DNA methylation alterations. Cox's proportional hazards models were used for univariate and multivariate analysis, and multiple pathological variables and molecular markers were adjusted.

### Real-Time Methylation-Specific PCR (MSP) Mastermix

Of available molecular techniques, the methylation-specific PCR (MSP) is effective for assessing DNA methylation levels. However, one of the major limitations of this technique is that conventional MSP assays can only analyze one amplicon at a time. For feasibility and efficient amplification of DNA templates by traditional PCR, ready-to-use solutions containing all the required reagents at optimal concentrations are commercially available. However, none is suitable for DNA methylation analysis. Due to the requirement of chemical pre-treatment step (bisulfite treatment) in methylation analysis, the DNA sample is left fragmented and in poor quality. A majority of the commercially-available solutions perform sub-optimal on the bisulfite-treated DNA samples. Thus, as described herein, a highly sensitive and robust mastermix was developed specifically for DNA methylation analysis in DNA samples extracted from various samples including archived patient samples (e.g., FFPE; fixed in formalin and embedded in paraffin).

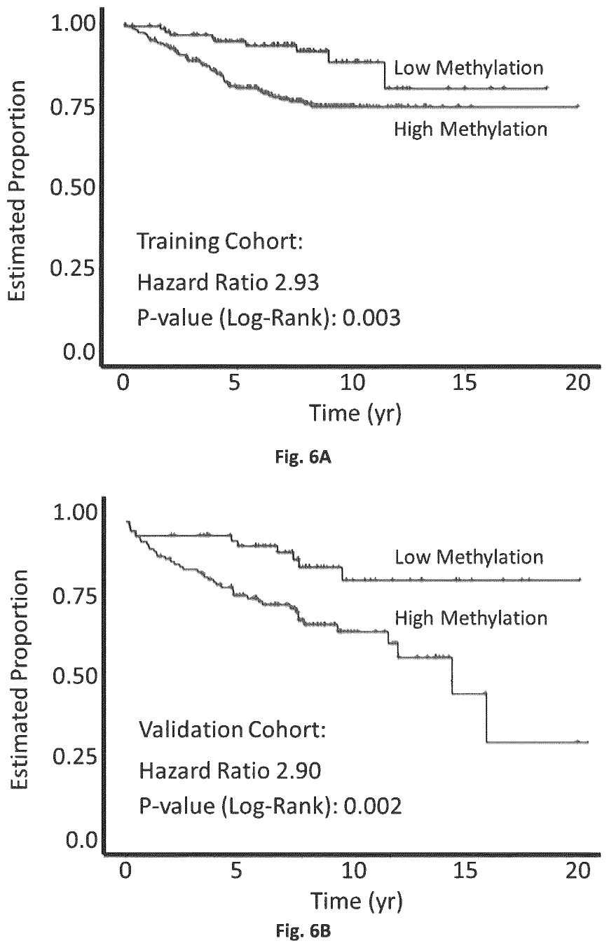

Mastermix (MMx) embodiments were formulated specifically to work with bisulfite-treated DNA samples. Over 15 different MSP assays were tested using the mastermix in singleplex format, all of which showed robust amplification from bisulfite treated DNA from archival tissue samples (FFPE). A representative amplification plot from a singleplex MSP assay is shown in FIG. 7A. For multiplexing purposes, MSP assay parameters were re-adjusted and FIG. 7B shows a typical amplification profile of one triplex MSP assays in which three separate DNA methylation regions were simultaneously detected via amplification. Formulations for singleplex and multiplex embodiments are shown in Table 8. The multiplex embodiment was tested with up to four gene targets, typical of what can be done with most real-time PCR instruments which come with four-six color channels. Thus the number of gene targets is limited by the capability of the PCR instrument used, i.e., the number of channels. It is expected that the multiplex embodiment will work with more than four gene targets with a suitable PCR instrument provided that the colours used for different targets don't cross-react or overlap.

TABLE 9
Formulations of New England Biolabs (NEB) mix and masterix
embodiments for singleplex and multiplex assays.

| | | | | | qMSP MMx | |
|---|---|---|---|---|---|---|
| | | | qMSP MMx | | (Multiplex up to 4 | |
| MASTER MIX: | NEB mix | | (Singleplex) | | gene targets) | |
| 5 X EpiMark Hot Start Taq | 1X | | 1X | | 1X | |
| Reaction Buffer | | | | | | |
| dNTPs | 200 | μM | 200 | μM | 200 | μM |
| | — | | 0-3.2 | mM | 0-3.2 | mM |
| Epimark Hot Start Taq DNA | 0.25 | Units | 0.25 | Units | 0.25 | Units |
| Polymerase | | | | | | |
| BSA | — | | 0.5 | mg/mL | 0.5 | mg/mL |
| ROX/MUSTANG purple dye | — | | ~24.5 | nM | ~24.5 | nM |
| (passive reference) | | | | | | |
| | <1000 | ng | 100 | pg-μM | 100 | pg-1 μM |

| | | | | | |
|---|---|---|---|---|---|
| Gene A Forward Primer | 0.2 μM (0.05-1 μM) | 0.4 μM | (0.05-1 μM) | 0.4 μM | (0.05-1 μM) |
| Gene A Reverse Primer | 0.2 μM (0.05-1 μM) | 0.4 μM | (0.05-1 μM) | 0.4 μM | (0.05-1 μM) |
| Gene A Probe (or SYBR Green) | — | 0.15 μM | (0.05-1 μM | 0.15 μM | (0.05-1 μM) |

| | | | | |
|---|---|---|---|---|
| Gene B Forward Primer | — | — | 0.4 μM | (0.05-1 μM) |
| Gene B Reverse Primer | — | — | 0.4 μM | (0.05-1 μM) |
| Gene B Probe | — | — | 0.15 μM | (0.05-1 μM) |
| Gene C Forward Primer | — | — | 0.4 μM | (0.05-1 μM) |
| Gene C Reverse Primer | — | — | 0.4 μM | (0.05-1 μM) |
| Gene C Probe | — | — | 0.15 μM | (0.05-1 μM) |
| Gene D Forward Primer | — | — | 0.4 μM | (0.05-1 μM) |
| Gene D Reverse Primer | — | — | 0.4 μM | (0.05-1 μM) |
| Gene D Probe | — | — | 0.15 μM | (0.05-1 μM) |

Referring to Table 9 it is noted deoxyribonucleotide triphosphate (dNTP) is provided at 250 μM, although a range of concentration such as 50-500 μM may be used. The reaction buffer may contain MgCl₂ in sufficient amounts such that additional MgCl₂ is not required, hence 0 mM is specified as the low end of the range. However, in most cases the amount of MgCl₂ in the reaction buffer is not sufficient and it is added up to 3.2 mM. The concentration of BSA specified is generally considered suitable for stabilizing the DNA polymerase and neutralizing (any) potential inhibitors. Other suitable concentrations may also be used. Since BSA does not participate in the reaction, a concentration close to that specified is expected to be appropriate.

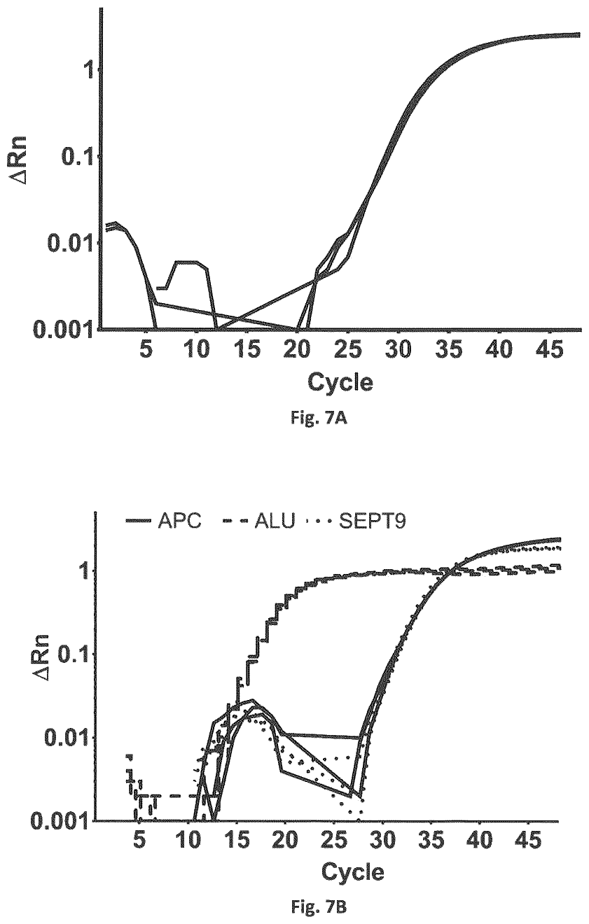

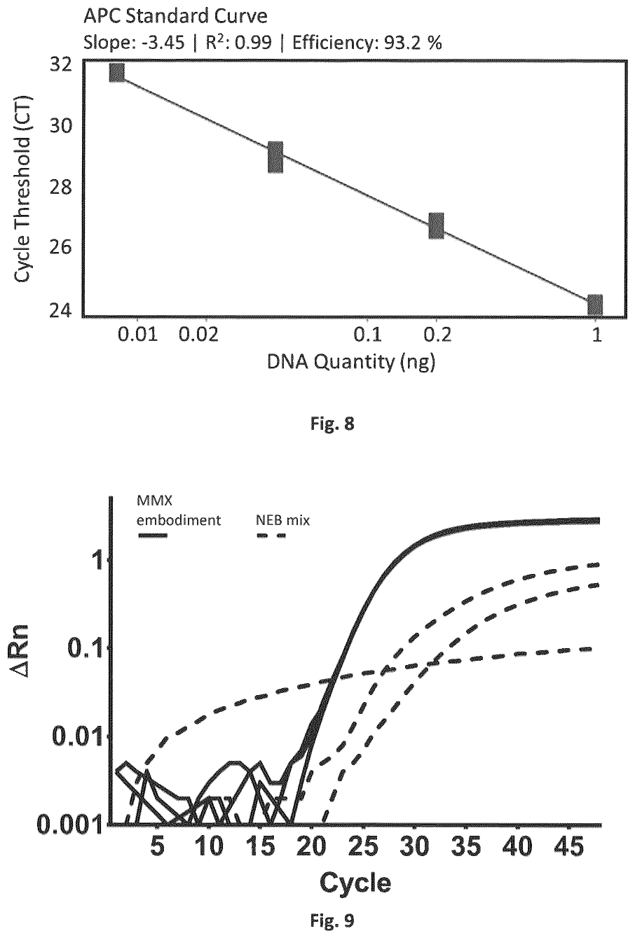

The performance of the APC MSP assay in multiplex reactions remained robust over several rounds of serial dilutions (FIG. 8). Using this mastermix embodiment, similar results were obtained with over 15 additional MSP assays. Performance was compared with a commercially available mix from New England Biolabs Ltd. (Whitby, ON, Canada) (NEB). As shown in FIG. 9, the mastermix embodiment worked with all of the MSP assays tests, whereas several MSP assays failed to amplify or showed poor reaction efficiency when used with the NEB mix. Characteristics of the mastermix embodiment as tested and the NEB mix are summarized in Table 10.

TABLE 10
Comparison of characteristics of a mastermix
embodiment and New England Biolabs (NEB) mix.

| Application | Mastermix | NEB mix |
|---|---|---|
| Bisulfite-treated DNA | ✓ | ✓ |
| Detection methodology | Real-time and routine | Endpoint/routine |
| No of targets | Up to 4 | 1 |
| Melt curve analysis | ✓ | ✓ |
| Tissue sample | Fresh/frozen and | Fresh/frozen |
| | archival (FFPE) | |
| Template with low copy | ✓ | No |
| numbers | | |

An exemplary protocol for using the mastermix is as follows:
1. Add components (for a singleplex or multiplex assay) according to Table 10 to a PCR reaction tube.
2. Gently mix the reaction. If using a plate, cover with optical adherent film and spin in PCR plate spinner for several seconds. Inspect wells to ensure liquid is collected at the bottom of each occupied well.
3. Transfer PCR tubes or reaction plate to a PCR thermocycler and program cycling according to the schedule in Table 11.

TABLE 11
PCR thermocycler program according to an MSP embodiment.

| Cycle Step | Temp | Time | |
|---|---|---|---|
| Step 1: 1 cycle | 95° C. | 30 | seconds |
| Step 2: 7 cycles | 95° C. | 30 | seconds |
| | 68° C. (−2° C. | 30 | seconds |
| | touchdown per cycle) | | |
| | 68° C. | 30 | seconds |
| Step 3: 48 cycles | 95° C. | 30 | seconds |
| | 58° C. | 30 | seconds |
| | 68° C. | 30 | seconds |
| Step 4: 1 cycle | 68° C. | 5 | minutes |

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

### REFERENCES

Amling, C. L., et al. (2001). Defining prostate specific antigen progression after radical prostatectomy: what is the most appropriate cut point? J. Urol. 165(4), 1146-51.
Aubry, W., Lieberthal, R., Willis, A., Bagley, G., Willis, S. M., and Layton, A. (2013). Budget Impact Model: Epigenetic Assay Can Help Avoid Unnecessary Repeated Prostate Biopsies and Reduce Healthcare Spending. Am. Health Drug Benefits 6, 15-24.
Bhasin, J. M., Lee, B. H., Matkin, L., Taylor, M. G., Hu, B., Xu, Y., Magi-Galluzzi, C., Klein, E. A., and Ting, A. H. (2015). Methylome-wide Sequencing Detects DNA Hypermethylation Distinguishing Indolent from Aggressive Prostate Cancer. Cell Rep. 13, 2135-2146.
Bhasin, J. M., Hu, B., and Ting, A. H. (2016). MethylAction: detecting differentially methylated regions that distinguish biological subtypes. Nucleic Acids Res. 44, 106-116.
Bonferroni, C. (1936). Teoria statistica delle classi e calcolo delle probabilita. Pubblicazioni R Ist. Super. Sci. Econ. E Commericiali Firenze 8, 3-62.
Cookson, M. S., et al. (2007). Variation in the definition of biochemical recurrence in patients treated for localized prostate cancer: the American Urological Association Prostate Guidelines for Localized Prostate Cancer Update Panel report and recommendations for a standard in the reporting of surgical outcomes. J. Urol. 177(2), 540-5.
Bustin, S. A., et al. (2009). The MIQE guidelines: minimum information for publication of quantitative real-time PCR experiments. Clin. Chem. 55(4), 611-622.
Fraser, M., Sabelnykova, V. Y., Yamaguchi, T. N., Heisler, L. E., Livingstone, J., Huang, V., Shiah, Y.-J., Yousif, F., Lin, X., Masella, A. P., et al. (2017). Genomic hallmarks of localized, non-indolent prostate cancer. Nature 541, 359-364.
Gaspar, J. M., and Hart, R. P. (2017). DMRfinder: efficiently identifying differentially methylated regions from MethylC-seq data. BMC Bioinformatics 18.
Haldrup, C., Mundbjerg, K., Vestergaard, E. M., Lamy, P., Wild, P., Schulz, W. A., Arsov, C.,
Visakorpi, T., Borre, M., Hoyer, S., et al. (2013). DNA methylation signatures for prediction of biochemical recurrence after radical prostatectomy of clinically localized prostate cancer. J. Clin. Oncol. Off. J. Am. Soc. Clin. Oncol. 31, 3250-3258.
Kamil Slowikowski (2017). ggrepel: Repulsive Text and Label Geoms for “ggplot2.”
Kang, G. H., Lee, S., Lee, H. J., and Hwang, K. S. (2004). Aberrant CpG island hypermethylation of multiple genes in prostate cancer and prostatic intraepithelial neoplasia. J. Pathol. 202, 233-240.
Lin, P.-C., Giannopoulou, E. G., Park, K., Mosquera, J. M., Sboner, A., Tewari, A. K., Garraway, L. A., Beltran, H., Rubin, M. A., and Elemento, 0. (2013). Epigenomic alterations in localized and advanced prostate cancer. Neoplasia N. Y. N 15, 373-383.
Mahapatra, S., Klee, E. W., Young, C. Y. F., Sun, Z., Jimenez, R. E., Klee, G. G., Tindall, D. J., and Donkena, K. V. (2012). Global methylation profiling for risk prediction of prostate cancer. Clin. Cancer Res. Off. J. Am. Assoc. Cancer Res. 18, 2882-2895.
Ngollo, M., Dagdemir, A., Karsli-Ceppioglu, S., Judes, G., Pajon, A., Penault-Llorca, F., Boiteux, J.-P., Bignon, Y.-J., Guy, L., and Bernard-Gallon, D. J. (2014). Epigenetic modifications in prostate cancer. Epigenomics 6, 415-426.
Olkhov-Mitsel, E., Zdravic, D., Kron, K., Kwast, T. van der, Fleshner, N., and Bapat, B. (2014). Novel Multiplex MethyLight Protocol for Detection of DNA Methylation in Patient Tissues and Bodily Fluids. Sci. Rep. 4, 4432.
Partin, A. W., Van Neste, L., Klein, E. A., Marks, L. S., Gee, J. R., Troyer, D. A., Rieger-Christ, K., Jones, J. S., Magi-Galluzzi, C., Mangold, L. A., et al. (2014). Clinical validation of an epigenetic assay to predict negative histopathological results in repeat prostate biopsies. J. Urol. 192, 1081-1087.
Patel, P. G., et al. (2016). Preparation of formalin-fixed paraffin-embedded tissue cores for both RNA and DNA extraction. J. Vis. Exp. 114, e54299.
Patel, P. G., et al. (2017). Reliability and performance of commercial RNA and DNA extraction kits for FFPE tissue cores. PLoS One 12(6), e0179732.
Perry, A. S., Watson, R. W. G., Lawler, M., and Hollywood, D. (2010). The epigenome as a therapeutic target in prostate cancer. Nat. Rev. Urol. 7, 668-680.
Pfaffl, M. W. (2001). A new mathematical model for relative quantification in real-time RT-PCR. Nucleic Acids Res. 29, e45.
R Core Team (2017). R: A Language and Environment for Statistical Computing (R Foundation for Statistical Computing).
Robin, X., Turck, N., Hainard, A., Tiberti, N., Lisacek, F., Sanchez, J.-C., and Müller, M. (2011). pROC: an open-source package for R and S+ to analyze and compare ROC curves. BMC Bioinformatics 12, 77.
Ruggero, K., Farran-Matas, S., Martinez-Tebar, A., and Aytes, A. (2018). Epigenetic Regulation in Prostate Cancer Progression. Curr. Mol. Biol. Rep. 4, 101-115.
Stephenson, A. J., et al. (2006). Defining biochemical recurrence of prostate cancer after radical prostatectomy: a proposal for a standardized definition. J. Clin. Oncol. 20; 24(24), 3973-8.
Stewart, G. D., Van Neste, L., Delvenne, P., Delrée, P., Delga, A., McNeill, S. A., O'Donnell, M., Clark, J., Van Criekinge, W., Bigley, J., et al. (2013). Clinical utility of an epigenetic assay to detect occult prostate cancer in histopathologically negative biopsies: results of the MATLOC study. J. Urol. 189, 1110-1116.
The Cancer Genome Atlas Research Network (2015). The molecular taxonomy of primary prostate cancer. Cell 163, 1011-1025.
Valdés-Mora, F., and Clark, S. J. (2015). Prostate cancer epigenetic biomarkers: next-generation technologies. Oncogene 34, 1609-1618.
Vanaja, D. K., Ehrich, M., Van den Boom, D., Cheville, J. C., Karnes, R. J., Tindall, D. J., Cantor, C. R., and Young, C. Y. F. (2009). Hypermethylation of genes for diagnosis and risk stratification of prostate cancer. Cancer Invest. 27, 549-560.
Wickham, H. (2009). ggplot2: Elegant Graphics for Data Analysis (New York: Springer-Verlag).
Wu, H., Xu, T., Feng, H., Chen, L., Li, B., Yao, B., Qin, Z., Jin, P., and Conneely, K. N. (2015). Detection of differentially methylated regions from whole-genome bisulfite sequencing data without replicates. Nucleic Acids Res. 43, e141.
Yegnasubramanian, S., Kowalski, J., Gonzalgo, M. L., Zahurak, M., Piantadosi, S., Walsh, P. C., Bova, G. S., De Marzo, A. M., Isaacs, W. B., and Nelson, W. G. (2004). Hypermethylation of CpG islands in primary and metastatic human prostate cancer. Cancer Res. 64, 1975-1986.

1. A method for detecting prostate cancer in a subject, comprising:
bisulfite converting genomic DNA obtained from the subject;
mixing the bisulfite-converted DNA with a mastermix for amplifying and/or sequencing the DNA;
wherein amplifying includes a selected region of each of GAS6, GSTP1, and HAPLN3 genes;
detecting hypermethylation of the selected regions of the GAS6, GSTP1, and HAPLN3 genes;
using the detected hypermethylation to identify prostate cancer in the subject.

2. The method of claim 1, comprising using probes comprising SEQ ID NOs. 23, 8, and 35, or functional equivalents thereof, for the GAS6, GSTP1, and HAPLN3 genes, respectively.

3. The method of claim 1, comprising subjecting the detected hypermethylation of the GAS6, GSTP1, and HAPLN3 genes to a classifier to identify prostate cancer in the subject.

4. The method of claim 1, wherein:
the selected hypermethylated region of the GAS6 gene is between a forward primer of SEQ ID NO: 22 and a reverse primer of SEQ ID NO: 24, or functional equivalents thereof;
the selected hypermethylated region of the GSTP1 gene is between a forward primer of SEQ ID NO: 7 and a reverse primer of SEQ ID NO: 9, or functional equivalents thereof; and
the selected hypermethylated region of the HAPLN3 gene is between a forward primer of SEQ ID NO: 34 and a reverse primer of SEQ ID NO: 36, or functional equivalents thereof.

5. The method of claim 1, wherein amplifying comprises using methylation-specific PCR (MSP).

6. The method of claim 5, wherein the mastermix comprises:
reaction buffer, 1×;
deoxyribonucleotide triphosphate (dNTP), 50-500 μM;
MgCl₂, 0-3.2 mM;
DNA polymerase, 0.25 units (U);
a concentration of BSA that stabilizes the DNA polymerase and neutralizes any potential inhibitors; and
ROX reference dye, 24.5 nM.

7. The method of claim 1, wherein amplifying and sequencing comprises using next generation sequencing (NGS).

8. The method of claim 1, wherein the genomic DNA is obtained from a biological sample selected from fresh/frozen prostate tissue, archival prostate tissue including formalin fixed and paraffin embedded (FFPE tissue), blood, and urine.

9. The method of claim 1,
wherein amplifying includes a selected region of each of GSTP1, CCDC181, HAPLN3, GSTM2, GAS6, RASSF1, and APC genes;
the method comprising detecting hypermethylation of the selected regions of the GSTP1, CCDC181, HAPLN3, GSTM2, GAS6, RASSF1, and APC genes; and
using the detected hypermethylation to identify prostate cancer in the subject.

10. The method of claim 9, comprising using probes comprising SEQ ID NOs. 8, 11, 35, 17, 23, 5, and 32, or functional equivalents thereof, for the GSTP1, CCDC181, HAPLN3, GSTM2, GAS6, RASSF1, and APC genes, respectively.

11. The method of claim 9, comprising subjecting the detected hypermethylation of the GSTP1, CCDC181, HAPLN3, GSTM2, GAS6, RASSF1, and APC genes to a classifier to identify prostate cancer in the subject.

12. The method of claim 9, wherein:
the selected hypermethylated region of the GSTP1 gene is between a forward primer of SEQ ID NO: 7 and a reverse primer of SEQ ID NO: 9, or functional equivalents thereof;
the selected hypermethylated region of the CCDC181 gene is between a forward primer of SEQ ID NO: 10 and a reverse primer of SEQ ID NO: 12, or functional equivalents thereof;
the selected hypermethylated region of the HAPLN3 gene is between a forward primer of SEQ ID NO: 34 and a reverse primer of SEQ ID NO: 36, or functional equivalents thereof;
the selected hypermethylated region of the GSTM2 gene is between a forward primer of SEQ ID NO: 16 and a reverse primer of SEQ ID NO: 18, or functional equivalents thereof;
the selected hypermethylated region of the GAS6 gene is between a forward primer of SEQ ID NO: 22 and a reverse primer of SEQ ID NO: 24, or functional equivalents thereof;
the selected hypermethylated region of the RASSF1 gene is between a forward primer of SEQ ID NO: 4 and a reverse primer of SEQ ID NO: 6, or functional equivalents thereof; and
the selected hypermethylated region of the APC gene is between a forward primer of SEQ ID NO: 31 and a reverse primer of SEQ ID NO: 33, or functional equivalents thereof.

13. The method of claim 9, wherein amplifying comprises using methylation-specific PCR (MSP).

14. The method of claim 13, wherein the mastermix comprises:
reaction buffer, 1×;
deoxyribonucleotide triphosphate (dNTP), 50-500 μM;
MgCl₂, 0-3.2 mM;
DNA polymerase, 0.25 units (U);
a concentration of BSA that stabilizes the DNA polymerase and neutralizes any potential inhibitors; and
ROX reference dye, 24.5 nM.

15. The method of claim 9, wherein amplifying and sequencing comprises using next generation sequencing (NGS).

16. The method of claim 9, wherein the genomic DNA is obtained from a biological sample selected from fresh/frozen prostate tissue, archival prostate tissue including formalin fixed and paraffin embedded (FFPE tissue), blood, and urine.

17. A method for identifying a prostate cancer patient at risk of developing biochemical recurrence, and/or suitable for treatment with an additional and/or alternative therapy, comprising:
bisulfite converting genomic DNA obtained from the subject;
mixing the bisulfite-converted DNA with a mastermix for amplifying and/or sequencing the DNA;
wherein amplifying includes a selected region in UCHL1 gene;
detecting hypermethylation of the selected region of the UCHL1 gene;
using the detected hypermethylation to identify risk of developing biochemical recurrence of prostate cancer, and/or suitability for treatment with an additional and/or alternative therapy.

18. The method of claim 17, comprising using a probe comprising SEQ ID NO. 44, or a functional equivalent thereof, for the UCHL1 gene, respectively.

19. The method of claim 17, wherein the selected hypermethylated region of the UCHL1 gene is between a forward primer of SEQ ID NO: 43 and a reverse primer of SEQ ID NO: 45, or a functional equivalent thereof.

20. The method of claim 17, wherein amplifying comprises using methylation-specific PCR (MSP).

21. The method of claim 20, wherein the mastermix comprises:
reaction buffer, 1×;
deoxyribonucleotide triphosphate (dNTP), 50-500 μM;
MgCl₂, 0-3.2 mM;
DNA polymerase, 0.25 units (U);
a concentration of BSA that stabilizes the DNA polymerase and neutralizes any potential inhibitors; and
ROX reference dye, 24.5 nM.

22. The method of claim 17, wherein amplifying and sequencing comprises using next generation sequencing (NGS).

23. The method of claim 17, wherein the genomic DNA is obtained from a biological sample selected from fresh/frozen prostate tissue, archival prostate tissue including formalin fixed and paraffin embedded (FFPE tissue), blood, and urine.

24. A mastermix for methylation-specific PCR (MSP), comprising:
reaction buffer, 1×;
deoxyribonucleotide triphosphate (dNTP), 50-500 μM;
MgCl₂, 0-3.2 mM;
DNA polymerase, 0.25 units (U);
a concentration of BSA that stabilizes the DNA polymerase and neutralizes any potential inhibitors; and
ROX reference dye, 24.5 nM.

25. The mastermix of claim 24, for use with genomic DNA, 50 ρg-1 μg.

26. The mastermix of claim 24, for use with bisulfite-converted genomic DNA, 50 ρg-1 μg.

27. The mastermix of claim 26 for a singleplex MSP, wherein:
a gene forward primer concentration is 0.05-1 μM;
a gene reverse primer concentration is 0.05-1 μM; and
a gene probe or SYBR green dye concentration is 0.05-1 μM.

28. The mastermix of claim 27, wherein:
the gene forward primer concentration is 0.4 μM;
the gene reverse primer concentration is 0.4 μM; and
the gene probe or SYBR green dye concentration is 0.15 μM.

29. The mastermix of claim 26 for a multiplex MSP, wherein, for each gene:
a gene forward primer concentration is 0.05-1 μM;
a gene reverse primer concentration is 0.05-1 μM; and
a gene probe concentration is 0.05-1 μM;
wherein the multiplex MSP comprises 2, 3, or 4 genes.

30. The mastermix of claim 29, wherein, for each gene:
the gene forward primer concentration is 0.4 μM;
the gene reverse primer concentration is 0.4 μM; and
the gene probe concentration is 0.15 μM.

31. The mastermix of claim 29, wherein a gene probe for a first gene is replaced with SYBR green dye.

32. The mastermix of claim 30, wherein a gene probe for a first gene is replaced with SYBR green dye.

33. An MSP method, comprising:
adding the following to the mastermix of claim 24:
bisulfite-converted DNA, 50 ρg-1 μg;
a gene forward primer, 0.05-1 μM;
a gene reverse primer, 0.05-1 μM; and
a gene probe or SYBR green dye, 0.05-1 μM;
mixing;
performing PCR cycles including:
heat to about 95° C. for about 30 seconds;
about seven cycles of about 95° C. for about 30 seconds, cool to about 68° C. with about −2° C. touchdown for about 30 seconds, and hold at about 68° C. for about 30 seconds;
about 48 cycles of about 95° C. for about 30 seconds, about 68° C. for about 30 seconds, and about 68° C. for about 30 seconds; and
one cycle of about 68° C. for about five minutes.

34. The MSP method of claim 33 for multiplex MSP, wherein, for each gene:
a gene forward primer concentration is 0.05-1 μM;
a gene reverse primer concentration is 0.05-1 μM; and
a gene probe concentration is 0.05-1 μM;
wherein the multiplex MSP comprises 2, 3, or 4 genes.

35. The MSP method of claim 33, wherein a gene probe for a first gene is replaced with SYBR green dye.

36. A kit for detecting prostate cancer comprising the mastermix of claim 24, primers and probes for a selected methylation site in each of GAS6, GSTP1, and HAPLN3 genes, and instructions for detecting prostate cancer.

37. A kit for detecting prostate cancer comprising the mastermix of claim 24, primers and probes for a selected methylation site in each of GSTP1, CCDC181, HAPLN3, GSTM2, GAS6, RASSF1, and APC genes, and instructions for detecting prostate cancer.

38. A kit for detecting aggressive prostate cancer, prostate cancer patients at risk of developing biochemical recurrence, and/or prostate cancer patients suitable for treatment with an additional and/or alternative therapy, comprising the mastermix of claim 24, primers and probes for a selected methylation site in USCHL1 gene, and instructions for use.


SEQUENCE LISTING (48 sequences)

SEQ ID NO: 1 - DNA, 18 bp, Artificial Sequence
  Synthesized
aaacgcccgccgttaata

SEQ ID NO: 2 - DNA, 28 bp, Artificial Sequence
  CCCAACTACTCTAACCGCGATAAACACT
cccaactactctaaccgcgataaacact

SEQ ID NO: 3 - DNA, 22 bp, Artificial Sequence
  Synthesized
ttcgtggagatgttggagattt

SEQ ID NO: 4 - DNA, 18 bp, Artificial Sequence
  Synthesized
gcgttgaagtcggggttc

SEQ ID NO: 5 - DNA, 24 bp, Artificial Sequence
  Synthesized
acaaacgcgaaccgaacgaaacca

SEQ ID NO: 6 - DNA, 24 bp, Artificial Sequence
  CCCGTACTTCGCTAACTTTAAACG
cccgtacttcgctaactttaaacg

SEQ ID NO: 7 - DNA, 18 bp, Artificial Sequence
  Synthesized
atgtttcggcgcgttagt

SEQ ID NO: 8 - DNA, 24 bp, Artificial Sequence
  Synthesized
tcgttgcgtatatttcgttgcggt

SEQ ID NO: 9 - DNA, 22 bp, Artificial Sequence
  Synthesized
acctttccctctttcccaaatc

SEQ ID NO: 10 - DNA, 21 bp, Artificial Sequence
  Synthesized
atcgatttttttcggtattta

SEQ ID NO: 11 - DNA, 21 bp, Artificial Sequence
  Synthesized
tcggtgtttgcgaagggttag

SEQ ID NO: 12 - DNA, 23 bp, Artificial Sequence
  Synthesized
gcgaatctctataacgttaataa

SEQ ID NO: 13 - DNA, 21 bp, Artificial Sequence
  Synthesized
gcggtggtttacgtttgtaat

SEQ ID NO: 14 - DNA, 22 bp, Artificial Sequence
  Synthesized
aaataatccgcccgcctcgacc

SEQ ID NO: 15 - DNA, 25 bp, Artificial Sequence
  Synthesized
aaataatctcgatctcctaacctca

SEQ ID NO: 16 - DNA, 22 bp, Artificial Sequence
  Synthesized
ggatcgggagaatcggatagaa

SEQ ID NO: 17 - DNA, 25 bp, Artificial Sequence
  Synthesized
aaacaaccgtcaacgactcctaccc

SEQ ID NO: 18 - DNA, 20 bp, Artificial Sequence
  Synthesized
acttaacccaacccgaaacg

SEQ ID NO: 19 - DNA, 23 bp, Artificial Sequence
  Synthesized
ctctaccctctctaaacctctca

SEQ ID NO: 20 - DNA, 25 bp, Artificial Sequence
  Synthesized
tgggtatggtgttggttgttgtgga

SEQ ID NO: 21 - DNA, 22 bp, Artificial Sequence
  Synthesized
tttggtttatttcgcggatgtt

SEQ ID NO: 22 - DNA, 22 bp, Artificial Sequence
  Synthesized
aacattcctaaccgaaataccg

SEQ ID NO: 23 - DNA, 18 bp, Artificial Sequence
  Synthesized
acaccgtcgaactcctaa

SEQ ID NO: 24 - DNA, 20 bp, Artificial Sequence
  Synthesized
cggtttcgttttgttaggtg

SEQ ID NO: 25 - DNA, 22 bp, Artificial Sequence
  Synthesized
cgcgcgattcgttgtttattag

SEQ ID NO: 26 - DNA, 24 bp, Artificial Sequence
  Synthesized
cggttaacgcgtagttggatggga

SEQ ID NO: 27 - DNA, 22 bp, Artificial Sequence
  Synthesized
cccaccttcgaaatccgaaata

SEQ ID NO: 28 - DNA, 17 bp, Artificial Sequence
  Synthesized
gttaggcggttagggcg

SEQ ID NO: 29 - DNA, 24 bp, Artificial Sequence
  Synthesized
cgtaggagagtgtgttgggtagac

SEQ ID NO: 30 - DNA, 16 bp, Artificial Sequence
  Synthesized
ccgaacgcctccatcg

SEQ ID NO: 31 - DNA, 20 bp, Artificial Sequence
  Synthesized
ggaagcggagagagaagtag

SEQ ID NO: 32 - DNA, 21 bp, Artificial Sequence
  Synthesized
aattcgttggatgcggattag

SEQ ID NO: 33 - DNA, 18 bp, Artificial Sequence
  Synthesized
gacgaactcccgacgaaa

SEQ ID NO: 34 - DNA, 22 bp, Artificial Sequence
  Synthesized
gttttcgtagtgttcggtttac

SEQ ID NO: 35 - DNA, 20 bp, Artificial Sequence
  Synthesized
tcggattttgttcgggaggt

SEQ ID NO: 36 - DNA, 18 bp, Artificial Sequence
  Synthesized
gaattcctcccttaccgc

SEQ ID NO: 37 - DNA, 24 bp, Artificial Sequence
  Synthesized
tttttttcgtaatagcggttttgt

SEQ ID NO: 38 - DNA, 25 bp, Artificial Sequence
  Synthesized
tcgtatttttatttttgttttcggg

SEQ ID NO: 39 - DNA, 23 bp, Artificial Sequence
  Synthesized
atccaaaacaatccctaaaaacg

SEQ ID NO: 40 - DNA, 22 bp, Artificial Sequence
  Synthesized
gggtgtttagggattagagagg

SEQ ID NO: 41 - DNA, 24 bp, Artificial Sequence
  Synthesized
tttgggttcgggtcgtttgttacg

SEQ ID NO: 42 - DNA, 21 bp, Artificial Sequence
  Synthesized
cgaactcaacaaccgaatcac

SEQ ID NO: 43 - DNA, 22 bp, Artificial Sequence
  Synthesized
cggcgagtgagattgtaaggtt

SEQ ID NO: 44 - DNA, 28 bp, Artificial Sequence
  Synthesized
ttcggtcgtattatttcgcgttgcgtac

SEQ ID NO: 45 - DNA, 23 bp, Artificial Sequence
  Synthesized
gaacgatcgcgaccaaataaata

SEQ ID NO: 46 - DNA, 18 bp, Artificial Sequence
  Synthesized
aattccaccgccccaaac

SEQ ID NO: 47 - DNA, 19 bp, Artificial Sequence
  Synthesized
atttggcggaaatttgtgc

SEQ ID NO: 48 - DNA, 19 bp, Artificial Sequence
  Synthesized
cggaagcgttcgggtaaag